(19) United States
(12) Patent Application Publication
(10) Pub. No.: US 2026/0295253 A1
(43) Pub. Date: Oct. 1, 2026

(54) APPARATUS AND METHOD FOR ASSESSING DEVICE FUNCTION OF A BILATERAL SENSORY SYSTEM

(71) Applicant: Cochlear Limited, Macquarie University, NSW (AU)

(72) Inventors: Nathan Isaacson, Balgowlah, NSW (AU); Jamon Windeyer, Sydney, NSW (AU)

(21) Appl. No.: 19/133,173

(22) PCT Filed: Nov. 21, 2023
(86) PCT No.: PCT/IB2023/061750

§ 371 Date: May 27, 2025

(51) Int. Cl. A61N 1/36; H04R 25/00

(52) U.S. Cl. CPC A61N 1/36038; H04R 25/305; H04R 2460/13

(57) ABSTRACT

An apparatus includes at least one first microphone configured to be worn on or within a recipient's body and configured to generate microphone signals indicative of ambient sound from an environment of the recipient. The apparatus further includes first circuitry configured to, in response to the microphone signals, to generate stimulation signals indicative of the ambient sound and configured to be received by the recipient's body to evoke a hearing percept by the recipient. The apparatus further includes second circuitry configured to generate first data indicative of a first comparison of the responses of the at least one first microphone and at least one second microphone to the ambient sound at a first time. The second circuitry is further configured to generate second data indicative of a second comparison of the responses of the at least one first microphone and the at least one second microphone to the ambient sound at a second time. The second time is subsequent to the first time. The second circuitry is further configured to generate a performance evaluation of at least one aspect of the apparatus in response to a third comparison of the first and second data.

### BACKGROUND

### Field

The present application relates generally to medical implants and/or other systems having active components (e.g., transducers; actuators; microphones; sensors) responsive to sensory stimuli from an ambient environment.

### Description of the Related Art

Medical devices have provided a wide range of therapeutic benefits to recipients over recent decades. Medical devices can include internal or implantable components/devices, external or wearable components/devices, or combinations thereof (e.g., a device having an external component communicating with an implantable component). Medical devices, such as traditional hearing aids, partially or fully-implantable hearing prostheses (e.g., bone conduction devices, mechanical stimulators, cochlear implants, etc.), pacemakers, defibrillators, functional electrical stimulation devices, and other medical devices, have been successful in performing lifesaving and/or lifestyle enhancement functions and/or recipient monitoring for a number of years.

The types of medical devices and the ranges of functions performed thereby have increased over the years. For example, many medical devices, sometimes referred to as “implantable medical devices,” now often include one or more instruments, apparatus, sensors, processors, controllers or other functional mechanical or electrical components that are permanently or temporarily implanted in a recipient. These functional devices are typically used to diagnose, prevent, monitor, treat, or manage a disease/injury or symptom thereof, or to investigate, replace or modify the anatomy or a physiological process. Many of these functional devices utilize power and/or data received from external devices that are part of, or operate in conjunction with, implantable components.

### SUMMARY

In one aspect disclosed herein, an apparatus comprises at least one first microphone configured to be worn on or within a recipient's body. The at least one first microphone is configured to generate microphone signals indicative of ambient sound from an environment of the recipient. The apparatus further comprises first circuitry configured to receive the microphone signals and, in response to the microphone signals, to generate stimulation signals configured to be received by the recipient's body to evoke a hearing percept by the recipient. The stimulation signals are indicative of the ambient sound. The apparatus further comprises second circuitry configured to generate first data indicative of a first comparison of a response of the at least one first microphone to the ambient sound at a first time to a response of at least one second microphone to the ambient sound at the first time. The second circuitry is further configured to generate second data indicative of a second comparison of a response of the at least one first microphone to the ambient sound at a second time to a response of the at least one second microphone of the device to the ambient sound at the second time. The second time is subsequent to the first time. The second circuitry is further configured to generate a performance evaluation of at least one aspect of the apparatus in response to a third comparison of the first data and the second data.

In another aspect disclosed herein, an apparatus comprises communication circuitry configured to receive first information from at least one first transducer and second information from at least one second transducer. The first information and the second information are indicative of signals from an environment of a recipient or from the recipient's body. The apparatus further comprises evaluation circuitry configured to, during a first time period, receive a first portion of the first information indicative of a response of the at least one first transducer to the signals during the first time period, receive a first portion of the second information indicative of a response of the at least one second transducer to the signals during the first time period, and generate a first comparison of the first portion of the first information and the first portion of the second information. The evaluation circuitry is further configured to, during a second time period subsequent to the first time period, receive a second portion of the first information indicative of a response of the at least one first transducer to the signals during the second time period, receive a second portion of the second information indicative of a response of the at least one second transducer to the signals during the second time period, and generate a second comparison of the second portion of the first information and the second portion of the second information. The evaluation circuitry is further configured to generate a third comparison of the first comparison and the second comparison.

In another aspect disclosed herein, a method comprises measuring and storing a first differential between responses of a first prosthesis and a second prosthesis to ambient excitations at a first time. The method further comprises measuring and storing at least one second differential between responses of the first prosthesis and the second prosthesis to ambient excitations at a second time subsequent to the first time. The method further comprises detecting a performance degradation of one of the first and second prostheses, said detecting comprising comparing the at least one first differential to the second differential.

In another aspect disclosed herein, an apparatus comprises a first device comprising at least one first transducer configured to respond to signals and a second device comprising at least one second transducer configured to respond to the signals. At least one of the first and second devices is configured to be worn on or implanted in a recipient's body. The apparatus further comprises first circuitry configured to generate first data indicative of a first comparison of a response of the at least one first transducer to the signals at a first time to a response of the at least one second transducer to the signals at the first time. The apparatus further comprises second circuitry configured to generate second data indicative of a second comparison of a response of the at least one first transducer to the signals at a second time to a response of the at least one second transducer to the signals at the second time. The second time is subsequent to the first time. The apparatus further comprises third circuitry configured to generate a performance evaluation of at least one aspect of the apparatus in response to a third comparison of the first data and the second data.

### BRIEF DESCRIPTION OF THE DRAWINGS

Implementations are described herein in conjunction with the accompanying drawings, in which:

FIG. 1A is a perspective view of an example cochlear implant auditory prosthesis implanted in a recipient in accordance with certain implementations described herein;

FIG. 1B is a perspective view of an example fully implantable middle ear implant auditory prosthesis implanted in a recipient in accordance with certain implementations described herein;

FIG. 1C schematically illustrate a portion of another example transcutaneous bone conduction auditory prosthesis implanted in a recipient in accordance with certain implementations described herein;

FIG. 1D schematically illustrate a portion of an example acoustic hearing prosthesis comprising a portion positioned at least partially within the ear canal of a recipient in accordance with certain implementations described herein;

FIGS. 2A and 2B schematically illustrate an example apparatus in accordance with certain implementations described herein;

FIG. 2C schematically illustrates an example information flow diagram in accordance with the two example apparatus in accordance with certain implementations described herein;

FIGS. 3A and 3B are flow diagrams of example methods for performing an initial test at time tₙ and a subsequent test at time tm respectively, in a performance evaluation of a sensory prosthesis system (e.g., bilateral acoustic prosthesis system) in accordance with certain implementations described herein; and

FIG. 4 is a flow diagram of an example method for detecting a performance degradation of one of the first and second sensory prostheses of a sensory prosthesis system (e.g., bilateral acoustic prosthesis system) in accordance with certain implementations described herein.

### DETAILED DESCRIPTION

Certain implementations described herein provide a self-contained performance testing function for sensory prostheses that are worn binaurally by the recipient. The performance testing function can provide ongoing monitoring of a bilateral acoustic prosthesis system over relatively long time periods (e.g., days; weeks; months) by comparing signal processing characteristics of the acoustic prostheses to one another. The signal processing characteristics are monitored passively by comparing the response difference between the two acoustic prostheses to ambient sounds at a first time period with the response difference between the two acoustic prostheses to ambient sounds at a second time period subsequent to the first time period. By utilizing the relative differences between the responses of the two acoustic prostheses to the same ambient sounds (e.g., as opposed to absolute differences), certain implementations provide the ability to identify changes and/or trends in performance of the microphones and/or sound processing circuitry over time (e.g., rather than mere pass/fail) and can detect failures other than a defective microphone (e.g., clogged microphone cover ports). Certain implementations provide the ability to run a test with no additional hardware apart from the two acoustic prostheses and to run a test with neither acoustic prosthesis having a reliable acoustic output. By relying on data from at least two acoustic prostheses, certain implementations can reduce the likelihood of errors in signal processing hardware or firmware causing false fails or false passes. Certain implementations provide more robust performance as compared to a similar comparison between two microphones on a single device (e.g., reducing the likelihood of equal deterioration in performance of both microphones, leading to a false pass).

The teachings detailed herein are applicable, in at least some implementations, to any type of medical system (e.g., implantable or wearable). For example, the medical system can sense or receive information from the ambient environment or from the recipient's body. The medical system of certain implementations utilizes a transducer assembly configured to provide stimulation signals (e.g., electrical stimulation signals; optical stimulation signals; electromagnetic stimulation signals; vibrational stimulation signals; sound stimulation signals) to the recipient's body in response to received information and/or control signals (e.g., implantable sensor prostheses; implantable stimulation system). For example, the medical system can comprise an auditory prosthesis system and/or an auditory hearing device configured to generate and apply stimulation signals (e.g., electrical and/or vibrational) that are perceived by the recipient as sounds (e.g., evoking a hearing percept). Such wearable transducer assemblies can include hearing aid devices configured to be placed at least partially within an ear canal of the recipient and/or consumer electronics devices (e.g., headphones) or other systems having a bilateral configuration. Such implantable transducer assemblies can include but are not limited to: electro-acoustic electrical/acoustic systems, cochlear implant devices, implantable hearing aid devices, middle ear implant devices, bone conduction devices (e.g., active bone conduction devices; passive bone conduction devices, percutaneous bone conduction devices; transcutaneous bone conduction devices), Direct Acoustic Cochlear Implant (DACI), middle ear transducer (MET), electro-acoustic implant devices, other types of auditory prosthesis devices (e.g., auditory brain stimulators), and/or combinations or variations thereof, or any other suitable hearing device and/or prosthesis system with or without one or more external components. Merely for ease of description, apparatus and methods disclosed herein are primarily described with reference to illustrative bilateral systems (e.g., auditory systems comprising two auditory devices concurrently on substantially opposite sides of the recipient's skull), but implementations can include any type of bilateral sensory prosthesis that can utilize the teachings detailed herein and/or variations thereof. Certain implementations described herein can be referred to as “partially implantable,” “semi-implantable,” “mostly implantable,” “fully implantable,” or “totally implantable” auditory prostheses.

The teachings detailed herein and/or variations thereof may also be used with a variety of other medical devices that provide a wide range of therapeutic benefits to recipients, patients, or other users. For example, other sensory prosthesis systems that are configured to evoke other types of neural or sensory (e.g., sight, tactile, smell, taste) percepts are compatible with certain implementations described herein, including but are not limited to: vestibular devices (e.g., vestibular implants), visual devices (e.g., bionic eyes), visual prostheses (e.g., retinal implants), somatosensory implants, and chemosensory implants. In some implementations, the teachings detailed herein and/or variations thereof can be utilized in other types of implantable medical devices beyond sensory prostheses. For example, apparatus and methods disclosed herein and/or variations thereof can be used with one or more of the following: sensors; cardiac pacemakers; drug delivery systems; defibrillators; functional electrical stimulation devices; catheters; brain implants; seizure devices (e.g., devices for monitoring and/or treating epileptic events); sleep apnea devices; electroporation; pain relief devices; etc. Implementations can include any type of medical system that can utilize the teachings detailed herein and/or variations thereof (e.g., systems that may benefit from having two devices in or on the recipient's body, each with one or more transducers making measurements of the same aspect of the ambient environment or of the recipient's body).

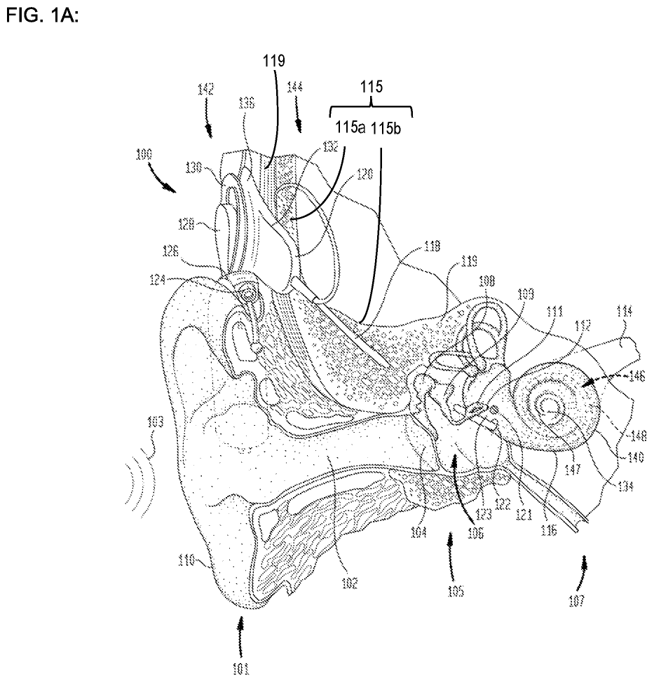

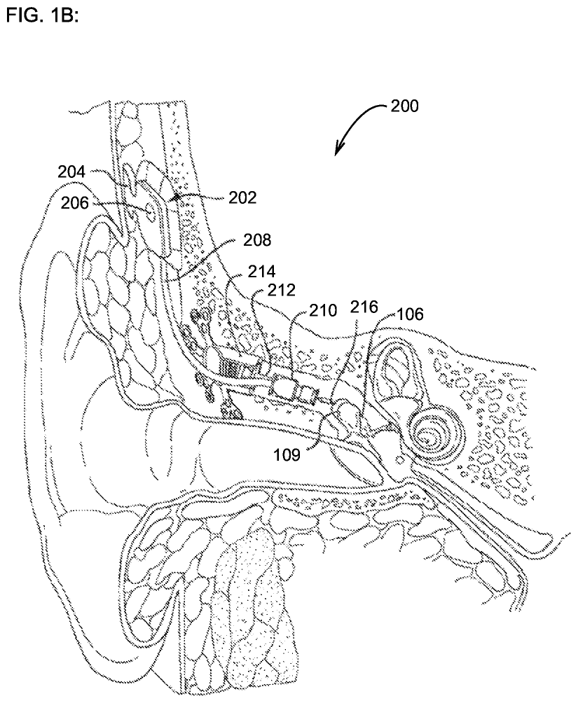

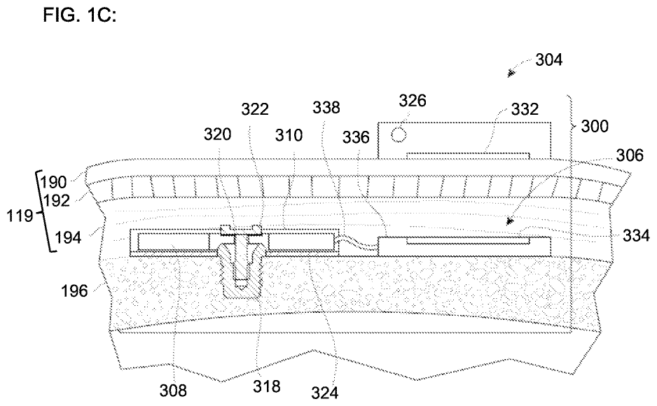

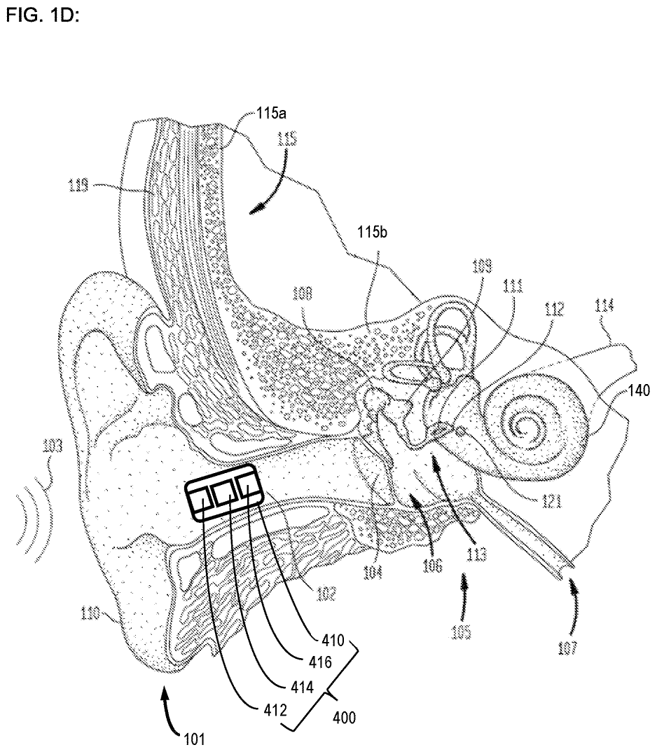

FIG. 1A is a perspective view of an example cochlear implant auditory prosthesis 100 implanted in a recipient in accordance with certain implementations described herein. The example auditory prosthesis 100 is shown in FIG. 1A as comprising an implanted stimulator unit 120 and a microphone assembly 124 that is external to the recipient (e.g., a partially implantable cochlear implant). An example auditory prosthesis 100 (e.g., a totally implantable cochlear implant; a mostly implantable cochlear implant) in accordance with certain implementations described herein can replace the external microphone assembly 124 shown in FIG. 1A with a subcutaneously implantable microphone assembly, as described more fully herein. In certain implementations, the example cochlear implant auditory prosthesis 100 of FIG. 1A can be in conjunction with a reservoir of liquid medicament as described herein.

As shown in FIG. 1A, the recipient has an outer ear 101, a middle ear 105, and an inner ear 107. In a fully functional ear, the outer ear 101 comprises an auricle 110 and an ear canal 102. An acoustic pressure or sound wave 103 is collected by the auricle 110 and is channeled into and through the ear canal 102. Disposed across the distal end of the ear canal 102 is a tympanic membrane 104 which vibrates in response to the sound wave 103. This vibration is coupled to oval window or fenestra ovalis 112 through three bones of middle ear 105, collectively referred to as the ossicles 106 and comprising the malleus 108, the incus 109, and the stapes 111 positioned in the middle ear cavity 113. The bones 108, 109, and 111 of the middle ear 105 serve to filter and amplify the sound wave 103, causing the oval window 112 to articulate, or vibrate in response to vibration of the tympanic membrane 104. This vibration sets up waves of fluid motion of the perilymph within cochlea 140. Such fluid motion, in turn, activates tiny hair cells (not shown) inside the cochlea 140. Activation of the hair cells causes appropriate nerve impulses to be generated and transferred through the spiral ganglion cells (not shown) and auditory nerve 114 to the brain (also not shown) where they are perceived as sound.

The human skull is formed from a number of different bones that support various anatomical features. Illustrated in FIG. 1A is the temporal bone 115 which is situated at the side and base of the recipient's skull (covered by a portion of the recipient's skin/muscle/fat, collectively referred to herein as tissue 119). For ease of reference, the temporal bone 115 is referred to herein as having a superior portion 115a and a mastoid portion 115b. The superior portion 115a comprises the section of the temporal bone 115 that extends superior to the auricle 110. That is, the superior portion 115a is the section of the temporal bone 115 that forms the side surface of the skull. The mastoid portion 115b, referred to herein simply as the mastoid 115b, is positioned inferior to the superior portion 115a. The mastoid 115b is the section of the temporal bone 115 that surrounds the middle ear 105.

As shown in FIG. 1A, the example auditory prosthesis 100 comprises one or more components which are temporarily or permanently implanted in the recipient. The example auditory prosthesis 100 is shown in FIG. 1A with an external component 142 which is directly or indirectly attached to the recipient's body, and an internal component 144 which is temporarily or permanently implanted in the recipient (e.g., positioned in a recess of the temporal bone adjacent auricle 110 of the recipient). The external component 142 typically comprises one or more sound input elements (e.g., an external microphone 124) for detecting sound, a sound processing unit 126 (e.g., disposed in a Behind-The-Ear unit), a power source (not shown), and an external transmitter unit 128. In the illustrative implementations of FIG. 1A, the external transmitter unit 128 comprises an external coil 130 (e.g., a wire antenna coil comprising multiple turns of electrically insulated single-strand or multi-strand platinum or gold wire) and, preferably, a magnet (not shown) secured directly or indirectly to the external coil 130. The external coil 130 of the external transmitter unit 128 is part of an inductive radio frequency (RF) communication link with the internal component 144. The sound processing unit 126 processes the output of the microphone 124 that is positioned externally to the recipient's body, in the depicted implementation, by the recipient's auricle 110. The sound processing unit 126 processes the output of the microphone 124 and generates encoded signals, sometimes referred to herein as encoded data signals, which are provided to the external transmitter unit 128 (e.g., via a cable). As will be appreciated, the sound processing unit 126 can utilize digital processing techniques to provide frequency shaping, amplification, compression, and other signal conditioning, including conditioning based on recipient-specific fitting parameters.

The power source of the external component 142 is configured to provide power to the auditory prosthesis 100, where the auditory prosthesis 100 includes a battery (e.g., located in the internal component 144, or disposed in a separate implanted location) that is recharged by the power provided from the external component 142 (e.g., via a transcutaneous energy transfer link). The transcutaneous energy transfer link is used to transfer power and/or data to the internal component 144 of the auditory prosthesis 100. Various types of energy transfer, such as infrared (IR), electromagnetic, capacitive, and inductive transfer, may be used to transfer the power and/or data from the external component 142 to the internal component 144. During operation of the auditory prosthesis 100, the power stored by the rechargeable battery is distributed to the various other implanted components as needed.

The internal component 144 comprises an internal receiver unit 132, a stimulator unit 120, and an elongate electrode assembly 118. In some implementations, the internal receiver unit 132 and the stimulator unit 120 are hermetically sealed within a biocompatible housing. The internal receiver unit 132 comprises an internal coil 136 (e.g., a wire antenna coil comprising multiple turns of electrically insulated single-strand or multi-strand platinum or gold wire), and preferably, a magnet (also not shown) fixed relative to the internal coil 136. The internal receiver unit 132 and the stimulator unit 120 are hermetically sealed within a biocompatible housing, sometimes collectively referred to as a stimulator/receiver unit. The internal coil 136 receives power and/or data signals from the external coil 130 via a transcutaneous energy transfer link (e.g., an inductive RF link). The stimulator unit 120 generates electrical stimulation signals based on the data signals, and the stimulation signals are delivered to the recipient via the elongate electrode assembly 118.

The elongate electrode assembly 118 has a proximal end connected to the stimulator unit 120, and a distal end implanted in the cochlea 140. The electrode assembly 118 extends from the stimulator unit 120 to the cochlea 140 through the mastoid bone 119. In some implementations, the electrode assembly 118 may be implanted at least in the basal region 116, and sometimes further. For example, the electrode assembly 118 may extend towards apical end of cochlea 140, referred to as cochlea apex 134. In certain circumstances, the electrode assembly 118 may be inserted into the cochlea 140 via a cochleostomy 122. In other circumstances, a cochleostomy may be formed through the round window 121, the oval window 112, the promontory 123, or through an apical turn 147 of the cochlea 140.

The elongate electrode assembly 118 comprises a longitudinally aligned and distally extending array 146 of electrodes or contacts 148, sometimes referred to as electrode or contact array 146 herein, disposed along a length thereof. Although the electrode array 146 can be disposed on the electrode assembly 118, in most practical applications, the electrode array 146 is integrated into the electrode assembly 118 (e.g., the electrode array 146 is disposed in the electrode assembly 118). As noted, the stimulator unit 120 generates stimulation signals which are applied by the electrodes 148 to the cochlea 140, thereby stimulating the auditory nerve 114.

While FIG. 1A schematically illustrates an auditory prosthesis 100 utilizing an external component 142 comprising an external microphone 124, an external sound processing unit 126, and an external power source, in certain other implementations, one or more of the microphone 124, sound processing unit 126, and power source are implantable on or within the recipient (e.g., within the internal component 144). For example, the auditory prosthesis 100 can have each of the microphone 124, sound processing unit 126, and power source implantable on or within the recipient (e.g., encapsulated within a biocompatible assembly located subcutaneously), and can be referred to as a totally implantable cochlear implant (“TICI”). For another example, the auditory prosthesis 100 can have most components of the cochlear implant (e.g., excluding the microphone, which can be an in-the-ear-canal microphone) implantable on or within the recipient, and can be referred to as a mostly implantable cochlear implant (“MICI”).

FIG. 1B schematically illustrates a perspective view of an example fully implantable auditory prosthesis 200 (e.g., fully implantable middle ear implant or totally implantable acoustic system), implanted in a recipient, utilizing an acoustic actuator in accordance with certain implementations described herein. The example auditory prosthesis 200 of FIG. 1B comprises a biocompatible implantable assembly 202 (e.g., comprising an implantable capsule) located subcutaneously (e.g., beneath the recipient's skin and on a recipient's skull). While FIG. 1B schematically illustrates an example implantable assembly 202 comprising a microphone, in other example auditory prostheses 200, a pendant microphone can be used (e.g., connected to the implantable assembly 202 by a cable). The implantable assembly 202 includes a signal receiver 204 (e.g., comprising a coil element) and an acoustic transducer 206 (e.g., a microphone comprising a diaphragm and an electret or piezoelectric transducer) that is positioned to receive acoustic signals through the recipient's overlying tissue. The implantable assembly 202 may further be utilized to house a number of components of the fully implantable auditory prosthesis 200. For example, the implantable assembly 202 can include an energy storage device and a signal processor (e.g., a sound processing unit). Various additional processing logic and/or circuitry components can also be included in the implantable assembly 202 as a matter of design choice.

For the example auditory prosthesis 200 shown in FIG. 1B, the signal processor of the implantable assembly 202 is in operative communication (e.g., electrically interconnected via a wire 208) with an actuator 210 (e.g., comprising a transducer configured to generate mechanical vibrations in response to electrical signals from the signal processor). In certain implementations, the example auditory prosthesis 100, 200 shown in FIGS. 1A and 1B can comprise an implantable microphone assembly, such as the microphone assembly 206 shown in FIG. 1B. For such an example auditory prosthesis 100, the signal processor of the implantable assembly 202 can be in operative communication (e.g., electrically interconnected via a wire) with the microphone assembly 206 and the stimulator unit of the main implantable component 120. In certain implementations, at least one of the microphone assembly 206 and the signal processor (e.g., a sound processing unit) is implanted on or within the recipient.

The actuator 210 of the example auditory prosthesis 200 shown in FIG. 1B is supportably connected to a positioning system 212, which in turn, is connected to a bone anchor 214 mounted within the recipient's mastoid process (e.g., via a hole drilled through the skull). The actuator 210 includes a connection apparatus 216 for connecting the actuator 210 to the ossicles 106 of the recipient. In a connected state, the connection apparatus 216 provides a communication path for acoustic stimulation of the ossicles 106 (e.g., through transmission of vibrations from the actuator 210 to the incus 109).

During normal operation, ambient acoustic signals (e.g., ambient sound) impinge on the recipient's tissue and are received transcutaneously at the microphone assembly 206. Upon receipt of the transcutaneous signals, a signal processor within the implantable assembly 202 processes the signals to provide a processed audio drive signal via wire 208 to the actuator 210. As will be appreciated, the signal processor may utilize digital processing techniques to provide frequency shaping, amplification, compression, and other signal conditioning, including conditioning based on recipient-specific fitting parameters. The audio drive signal causes the actuator 210 to transmit vibrations at acoustic frequencies to the connection apparatus 216 to affect the desired sound sensation via mechanical stimulation of the incus 109 of the recipient.

The subcutaneously implantable microphone assembly 202 is configured to respond to auditory signals (e.g., sound; pressure variations in an audible frequency range) by generating output signals (e.g., electrical signals; optical signals; electromagnetic signals) indicative of the auditory signals received by the microphone assembly 202, and these output signals are used by the auditory prosthesis 100, 200 to generate stimulation signals which are provided to the recipient's auditory system. To compensate for the decreased acoustic signal strength reaching the microphone assembly 202 by virtue of being implanted, the diaphragm of an implantable microphone assembly 202 can be configured to provide higher sensitivity than are external non-implantable microphone assemblies. For example, the diaphragm of an implantable microphone assembly 202 can be configured to be more robust and/or larger than diaphragms for external non-implantable microphone assemblies.

FIG. 1C schematically illustrate a portion of an example transcutaneous bone conduction auditory prosthesis 300 implanted in a recipient in accordance with certain implementations described herein. As schematically illustrated by FIG. 1C, the example transcutaneous bone conduction auditory prosthesis 300 comprises an external device 304 and an implantable component 306. The auditory prosthesis 300 is an active transcutaneous bone conduction auditory prosthesis in that the vibrating actuator 308 is located in the implantable component 306. For example, a vibratory element in the form of a vibrating actuator 308 is located in a housing 310 of the implantable component 306. In certain implementations, the vibrating actuator 308 is a device that converts electrical signals into vibration. The vibrating actuator 308 can be in direct contact with the outer surface of the recipient's bone 196 (e.g., the vibrating actuator 308 is in substantial contact with the recipient's bone 196 such that vibration forces from the vibrating actuator 308 are communicated from the vibrating actuator 308 to the recipient's bone 196). In certain implementations, there can be one or more thin non-bone tissue layers (e.g., a silicone layer 324) between the vibrating actuator 308 and the recipient's bone 196 (e.g., bone tissue; skull bone) while still permitting sufficient support so as to allow efficient communication of the vibration forces generated by the vibrating actuator 308 to the recipient's bone 196.

In certain implementations, the external component 304 includes a sound input element 326 that converts sound into electrical signals. Specifically, the auditory prosthesis 300 provides these electrical signals to the vibrating actuator 308, or to a sound processor (not shown) that processes the electrical signals, and then provides those processed signals to the implantable component 306 through the tissue of the recipient (e.g., skin 190, fat 192, muscle 194) via a magnetic inductance link. For example, a communication coil 332 of the external component 304 can transmit these signals to an implanted communication coil 334 located in a housing 336 of the implantable component 306. Components (not shown) in the housing 336, such as, for example, a signal generator or an implanted sound processor, then generate electrical signals to be delivered to the vibrating actuator 308 via electrical lead assembly 338. The vibrating actuator 308 converts the electrical signals into vibrations. In certain implementations, the vibrating actuator 308 can be positioned with such proximity to the housing 336 that the electrical leads 338 are not present (e.g., the housing 310 and the housing 336 are the same single housing containing the vibrating actuator 308, the communication coil 334, and other components, such as, for example, a signal generator or a sound processor).

In certain implementations, the vibrating actuator 308 is mechanically coupled to the housing 310. The housing 310 and the vibrating actuator 308 collectively form a vibrating element. The housing 310 can be substantially rigidly attached to a bone fixture 318.

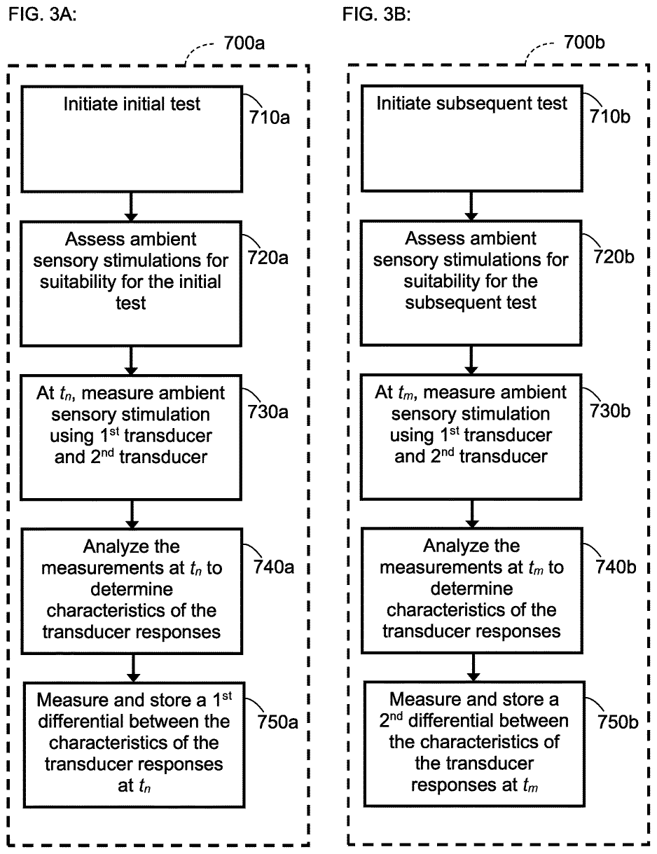

In this regard, the housing 310 can include a through hole 320 that is contoured to the outer contours of the bone fixture 318. The screw 322 can be used to secure the housing 310 to the bone fixture 318. As can be seen in FIG. 3, the head of the screw 322 is larger than the through hole 320 of the housing 310, and thus the screw 322 positively retains the housing 310 to the bone fixture 318. A portion of the screw 322 interfaces with the bone fixture 318, thus permitting the screw 322 to readily fit into an existing bone fixture 318 used in a percutaneous bone conduction device (or an existing passive bone conduction device). In certain implementations, the screw 322 is configured so that the same tools and procedures that are used to install and/or remove an abutment screw from the bone fixture 318 can be used to install and/or remove the screw 322 from the bone fixture 318.

The bone fixture 318 can be made of any material that has a known ability to integrate into surrounding bone tissue (e.g., comprising a material that exhibits acceptable osseointegration characteristics). In certain implementations, the bone fixture 318 is formed from a single piece of material (e.g., titanium) and comprises outer screw threads forming a male screw which is configured to be installed into the skull bone 196 and a flange configured to function as a stop when the fixture 318 is implanted into the skull bone 196. The screw threads can have a maximum diameter of about 3.5 mm to about 5.0 mm, and the flange can have a diameter which exceeds the maximum diameter of the screw threads (e.g., by approximately 10%-20%). The flange can have a planar bottom surface for resting against the outer bone surface, when the fixture 318 has been screwed down into the skull bone 196. The flange prevents the fixture 318 (e.g., the screw threads) from potentially completely penetrating completely through the bone 196.

The body of the fixture 318 can have a length sufficient to securely anchor the fixture 318 to the skull bone 196 without penetrating entirely through the skull bone 196. The length of the body can therefore depend on the thickness of the skull bone 196 at the implantation site. For example, the fixture 318 can have a length, measured from the planar bottom surface of the flange to the end of the distal region (e.g., the portion farthest from the flange), that is no greater than 5 mm or between about 3.0 mm to about 5.0 mm, which limits and/or prevents the possibility that the fixture 318 might go completely through the skull bone 196. The interior of the fixture 318 can further include an inner lower bore having female screw threads configured to mate with male screw threads of the screw 320 to the fixture 318. The fixture 318 can further include an inner upper bore that receives a bottom portion of the abutment 312.

FIG. 1D schematically illustrate a portion of an example auditory prosthesis 400 (e.g., hearing aid) comprising a portion 410 (e.g., housing) positioned at least partially within the ear canal 102 of a recipient in accordance with certain implementations described herein. Hearing aids rely on principles of air conduction to transmit amplified acoustic signals to the tympanic membrane 104, through the normal middle ear mechanisms to the cochlea 140, resulting in the increased perception of sound by the recipient. Typically, a hearing aid is positioned in the ear canal 102 or on the outer ear 110 to amplify received sound. It is worth noting that hearing aids are commonly referred to as hearing instruments by the industry and the receiver is the speaker that outputs sound to the recipient and not the microphone. As schematically illustrated by FIG. 1D, the example auditory prosthesis 400 comprises at least one microphone 412 (e.g., positioned outside the ear or within the cavity of the outer ear 101 or auricle 110, as schematically illustrated by FIG. 1D) configured to receive ambient sound 103, sound processing circuitry 414, and at least one speaker 416 (e.g., positioned within the ear canal 102, as schematically illustrated by FIG. 1D) configured to generate acoustic signals (e.g., amplified sound) that are transmitted to the tympanic membrane 104.

The example auditory prostheses 100 shown in FIG. 1A utilizes an external microphone 124, the auditory prosthesis 200 shown in FIG. 1B utilizes an implantable microphone assembly 206 comprising a subcutaneously implantable acoustic transducer, the example transcutaneous bone conduction auditory prosthesis 300 of FIG. 1C comprises an external sound input element 326 (e.g., external microphone), and the in-the-ear hearing aid 400 comprises an external microphone 412. In certain implementations described herein, a subcutaneously implantable sound input assembly (e.g., implanted microphone) is used with the auditory prostheses 100, 200, 300, 400 and/or one or more external microphone assemblies is used with the auditory prostheses 100, 200, 300, 400. In certain implementations, an external microphone assembly can be used to supplement an implantable microphone assembly of the auditory prosthesis 100, 200, 300, 400. Thus, the teachings detailed herein and/or variations thereof can be utilized with any type of external and/or implantable microphone arrangement, and the acoustic prostheses 100, 200, 300, 400 shown in FIGS. 1A-1D are merely illustrative.

With limited options for automatically measuring, diagnosing, and/or troubleshooting errors in the operation of an acoustic prosthesis, changes in performance (e.g., gradual changes, such a buildup of contaminants on the microphone protection material, which may not be readily apparent to the recipient) can progress to acoustically significant levels before the problem is noticed and rectified. Certain implementations described herein provide a fully or partially automated test that can be run by the acoustic prostheses of a bilateral acoustic prosthesis system in which the characteristics of the sounds from the ambient environment detected by at least two microphones (e.g., at least one microphone of each of the two acoustic prostheses; at least two microphones of one of the two acoustic prostheses) are compared to each other. The testing can be performed between fitting sessions and/or inspections of the acoustic prostheses by a practitioner (e.g., clinician). By monitoring changes in the differences of these characteristics over time, problems with at least one of the acoustic prostheses can be detected. This information can be relayed to the recipient and/or the practitioner and can be used to trigger further troubleshooting (e.g., changing microphone covers or scheduling a fitting session).

Certain implementations described herein provide better hearing performance (e.g., more quickly recognizing and rectifying reductions of hearing performance), more effective remote fitting sessions (e.g., by providing more detailed information about the device function for counseling or troubleshooting), reduced need for in-person fitting sessions (e.g., thereby improving convenience and clinic efficiency by reducing clinician time per recipient), and/or greater confidence in the proper functioning of the acoustic prosthesis system.

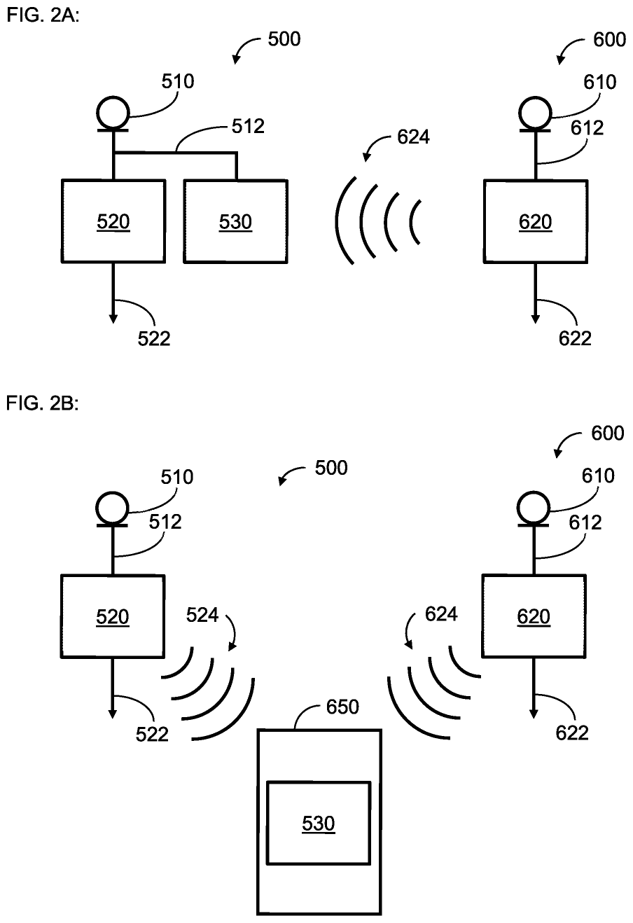

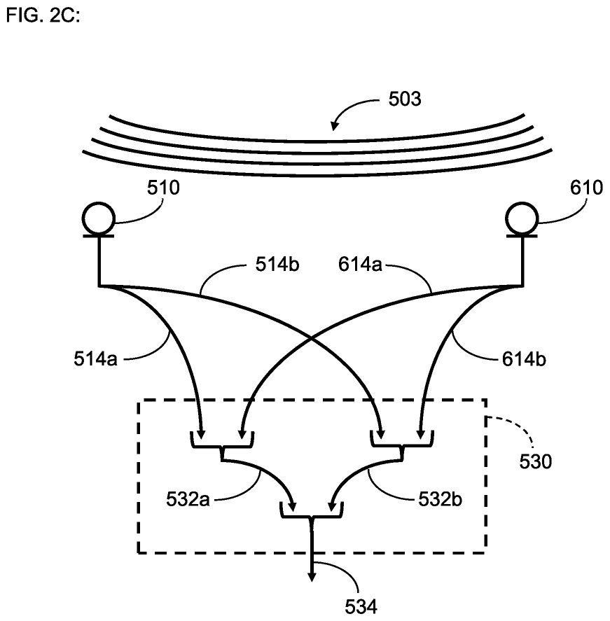

FIGS. 2A and 2B schematically illustrate two example apparatus 500 in accordance with certain implementations described herein. FIG. 2C schematically illustrates an example information flow diagram in accordance with the two example apparatus 500 in accordance with certain implementations described herein. The apparatus 500 comprises at least one first transducer 510 (e.g., microphone) configured to be worn on or within a recipient's body. The at least one first transducer 510 is configured to generate transducer signals 512 (e.g., microphone signals) indicative of ambient sensory excitations 503 (e.g., ambient sound 103; not shown in FIGS. 2A and 2B) from an environment of the recipient. The apparatus 500 further comprises first circuitry 520 configured to receive the transducer signals 512 and, in response to the transducer signals 512, to generate stimulation signals 522 configured to be received by the recipient's body to evoke a sensory (e.g., hearing) percept by the recipient. The stimulation signals 522 are indicative of the ambient sensory excitations 503. The apparatus 500 further comprises second circuitry 530 configured to generate first data 532a indicative of a first comparison of a response of the at least one first transducer 510 to the ambient sensory excitations 503 at a first time t₁ (e.g., first information 514a) to a response of at least one second transducer 610 (e.g., microphone) to the ambient sensory excitations 503 at the first time t₁ (e.g., second information 614a). The second circuitry 530 is further configured to generate second data 532b indicative of a second comparison of a response of the at least one first transducer 510 to the ambient sensory excitations 503 at a second time t₂ (e.g., first information 514b) to a response of the at least one second transducer 610 of the device 600 to the ambient sensory excitations 503 at the second time t₂ (e.g., second information 614b). The second time t₂ is subsequent to the first time t₁. The second circuitry 530 is further configured to generate a performance evaluation 534 of at least one aspect of the apparatus 500 in response to a third comparison of the first data 532a and the second data 532b.

In certain implementations, the at least one first transducer 510 and the first circuitry 520 (e.g., first stimulation circuitry) are components of a first sensory (e.g., auditory) prosthesis in operable communication with a first sensory subsystem (e.g., first ear) of the recipient and the at least one second transducer 610 is a component of a second sensory (e.g., auditory) prosthesis in operable communication with a second sensory subsystem (e.g., second ear) of the recipient. For example, the first auditory prosthesis and the second auditory prosthesis can be portions of a binaural or bilateral auditory prosthesis system implanted and/or worn by the recipient.

In certain implementations, the at least one first transducer 510 and the at least one second transducer 610 are components of the same apparatus 500 (e.g., an implanted microphone and a non-implanted microphone of a single acoustic prosthesis). In certain other implementations, the at least one second transducer 610 is a component of a device 600 separate from the apparatus 500 that comprises the at least one first transducer 510. As schematically illustrated in FIGS. 2A and 2B, the device 600 can comprise the at least one second transducer 610 (e.g., microphone) configured to generate second transducer signals 612 indicative of the ambient sensory excitations 503 from the environment of the recipient, and second stimulation circuitry 620 configured to receive the second transducer signals 612 and, in response to the second transducer signals 612, to generate second stimulation signals 622 configured to be received by the recipient's body to evoke a sensory (e.g., hearing) percept by the recipient. Both the stimulation signals 522 and the second stimulation signals 622 can be indicative of the ambient sensory excitations 503. More generally, the first sensory prosthesis (e.g., first auditory prosthesis) and the second sensory prosthesis (e.g., second auditory prosthesis) generate first information 514a and second information 614a, respectively, that are indicative of the same ambient sensory excitations 503 from an environment of the recipient at the first time t₁ and generate first information 514b and second information 614b, respectively, that are indicative of the same ambient sensory excitations 503 from the environment of the recipient at the second time t₂.

In certain implementations, the at least one first and second transducers 510, 610 each comprise an active component responsive to sensory stimuli from an ambient environment. While the at least one first and second transducers 510, 610 are described herein with regard to microphones that are components of auditory prostheses, the at least one first and second transducers 510, 610 can be components of other types of sensory prostheses. For example, the ambient sensory excitations 503 can be accelerations (e.g., the at least one first and second transducers 510, 610 each comprising an accelerometer for vestibular implants). For another example, the ambient sensory excitations 503 can be visual color/brightness data (e.g., the at least one first and second transducers 510, 610 each comprising a camera for visual implants). In certain implementations, the at least one first and second transducers 510, 610 can be components of monitoring devices configured to measure ambient signals without the measurements being used to stimulate the recipient's body. For example, the at least one first and second transducers 510, 610 can each comprise a sensor configured to make measurements of signals from the recipient's body (e.g., heart rate; blood pressure; blood glucose level; respiratory rate) to be recorded (e.g., stored).

In certain implementations, the first sensory prosthesis and/or the second sensory prosthesis comprises a cochlear implant (e.g., auditory prosthesis 100) configured to provide the stimulation signals 522, 622 (e.g., electrical signals) to a corresponding cochlea 140 of the recipient (see, e.g., FIG. 1A). In certain implementations, the first sensory prosthesis and/or the second sensory prosthesis comprises a fully implantable sensory prosthesis (e.g., auditory prosthesis 200 comprising an acoustic actuator 210) configured to provide the stimulation signals 522, 622 (e.g., vibrational signals) to a corresponding ossicle 106 (e.g., malleus 108; incus 109; stapes 111) of the recipient (see, e.g., FIG. 1B). In certain implementations, the first sensory prosthesis and/or the second sensory prosthesis comprises a bone conduction auditory prosthesis (e.g., auditory prosthesis 300) configured to provide the stimulation signals 522, 622 (e.g., vibrational signals) to a corresponding temporal bone portion 115 of the recipient (see, e.g., FIG. 1C). In certain implementations, the first sensory prosthesis and/or the second sensory prosthesis comprises a hearing aid (e.g., auditory prosthesis 400) configured to provide the stimulation signals 522, 622 (e.g., amplified sound signals) to a corresponding tympanic membrane 104 of the recipient.

In certain implementations, the first sensory prosthesis and the second sensory prosthesis are the same type of sensory prostheses (e.g., both cochlear implants; both having acoustic actuators; both bone conduction device; both hearing aids), while in certain other implementations, the first sensory prosthesis and the second sensory prosthesis are different types of sensory prostheses but are responsive to the same ambient sensory excitations (e.g., a bimodal system, an example of which is a hearing aid on one ear and a cochlear implant on the other ear). In certain implementations, the at least one first transducer 510 and the at least one second transducer 610 are both in operative communication with the same sensory prosthesis (e.g., the first transducer 510 comprising an implanted microphone and the second transducer 610 comprising an external microphone), while in certain other implementations, the at least one first transducer 510 is a component of a first sensory prosthesis and the at least one second transducer 610 is a component of a second sensory prosthesis different from the first sensory prosthesis. In certain implementations, the first and second sensory prostheses are worn or within opposite ears of the recipient's body, while in certain other implementations, the first and second sensory prostheses are worn or within the same ear of the recipient's body.

In certain implementations, the first circuitry 520 comprises at least one microcontroller that can comprise at least one application-specific integrated circuit (ASIC) microcontroller, digital signal processing (DSP) microcontroller, generalized integrated circuits programmed by software with computer executable instructions, and/or microcontroller core. The first circuitry 520 of certain implementations is a component of a sensory processing unit (e.g., sound processing unit 126) of the first sensory prosthesis. For example, the sound processing unit 126 of FIG. 1A is a behind-the-ear (BTE) sound processing unit configured to be attached to, and worn adjacent to, the recipient's ear. However, in certain other implementations, the sensory processing unit has other arrangements, such as by an off-the-ear (OTE) processing unit (e.g., a component having a generally cylindrical shape and which is configured to be magnetically coupled to the recipient's head), etc., a mini or micro-BTE unit, an in-the-canal unit that is configured to be located in the recipient's ear canal 102, a body-worn sensory processing unit, etc. Thus, the first circuitry 520 and the second stimulation circuitry 620 generate the first information 514a,b and second information 614a,b, respectively, that are both indicative of the same ambient sensory excitation 503 (e.g., ambient sound 103) from an environment of the recipient.

In certain implementations, the second circuitry 530 comprises at least one microcontroller that can comprise at least one application-specific integrated circuit (ASIC) microcontroller, digital signal processing (DSP) microcontroller, generalized integrated circuits programmed by software with computer executable instructions, and/or microcontroller core. In certain implementations, the second circuitry 530 and the first circuitry 520 comprise different portions of the same circuitry (e.g., a single microcontroller), while in certain other implementations, the second circuitry 530 and the first circuitry 520 comprise portions of different microcontrollers. In certain implementations, the second circuitry 530 comprises and/or is in operative communication with storage circuitry configured to store information (e.g., data; commands) accessed by the second circuitry 530 during operation (e.g., while providing the functionality of certain implementations described herein). The storage circuitry can comprise at least one tangible (e.g., non-transitory) computer readable storage medium, examples of which include but are not limited to: read only memory (ROM); random access memory (RAM); magnetic disk storage media; optical storage media; flash memory. The storage circuitry can be encoded with software (e.g., a computer program downloaded as an application) comprising computer executable instructions for instructing the second circuitry 530 (e.g., executable data access logic, evaluation logic, and/or information outputting logic). In certain implementations, the second circuitry 530 executes the instructions of the software to provide functionality as described herein. The second circuitry 530 of certain implementations further comprises other digital circuitry (e.g., registers; filters; output controllers; memory controllers).

In certain implementations, as schematically illustrated by FIG. 2A, the second circuitry 530 is a component of the first sensory prosthesis and is in electrical communication with the at least one first transducer 510 to receive information (e.g., the transducer signals 512 from the at least one first transducer 510). In certain other implementations, the second circuitry 530 receives the information from the first circuitry 520. The second circuitry 530 is also in wireless communication with the device 600 (e.g., the second sensory prosthesis) and configured to receive information 614 (e.g., wireless signals 624) from the device 600, the wireless signals 624 indicative of the second transducer signals 612. For example, the first and second sensory prostheses can each comprise communication circuitry configured to form a wireless data link (e.g., WiFi; Bluetooth; cellphone connection; telephony; or other Internet connection) across which the wireless signals 624 are transmitted from the device 600 to the second circuitry 530. In certain other implementations, the first and second sensory prostheses are in wired communication with one another (e.g., signals indicative of the second transducer signals 612 are transmitted from the device 600 to the second circuitry 530 via wires). For example, the first and second sensory prostheses can be portions of a non-surgical bimodal hearing system that comprises a single head-worn device that includes the at least one first transducer 510 and the at least one second transducer 610 on substantially opposite sides of the recipient's head.

In certain implementations (see, e.g., FIG. 2A), the second circuitry 530 is configured to generate the first data 532a by receiving a first portion of the transducer signals 512 (e.g., first information 514a; microphone signals generated in response to the ambient sound 103 at the first time t₁) and receiving a first portion of the wireless signals 624 (e.g., second information 614a; the second transducer signals 612) indicative of a response of the at least one second transducer 610 at the first time t₁ (e.g., microphone signals generated in response to the ambient sound 103 at the first time t₁). Similarly, the second circuitry 530 can be configured to generate the second data 532b by receiving a second portion of the transducer signals 512 (e.g., first information 514b; microphone signals generated in response to the ambient sound 103 at the second time t₂) and receiving a second portion of the wireless signals 624 (e.g., second information 614b; the second transducer signals 612) indicative of a response of the at least one second transducer 610 at the second time t₂ (e.g., microphone signals generated in response to the ambient sound 103 at the second time t₂).

In certain other implementations, as schematically illustrated by FIG. 2B, the second circuitry 530 is a component of a device 650 (e.g., smartphone, smart tablet, smart watch, computer, or other remote device operated by the recipient and/or a practitioner such as a clinician) separate from but in communication with both the first sensory prosthesis and the second sensory prosthesis. For example, the device 650 can comprise communication circuitry configured to wirelessly receive first information 514a,b (e.g., first portion of wireless signals 524) from a first sensory prosthesis and second information 614a,b (e.g., second portion of wireless signals 624) from a second sensory prosthesis. The communication circuitry of the device 650 and communication circuitry of the first sensory prosthesis can form a first wireless data link (e.g., WiFi; Bluetooth; cellphone connection; telephony; or other Internet connection) across which the wireless signals 524 indicative of the transducer signals 512 are transmitted from the first sensory prosthesis to the second circuitry 530. In addition, the communication circuitry of the device 650 and communication circuitry of the second sensory prosthesis can form a second wireless data link (e.g., WiFi; Bluetooth; cellphone connection; telephony; or other Internet connection) across which the wireless signals 624 are transmitted from the device 600 to the second circuitry 530.

In certain implementations (see, e.g., FIG. 2B), the second circuitry 530 is configured to, during a first time period, receive a first portion of information 514a (e.g., wireless signals 524) indicative of the response of the first sensory prosthesis, to receive a first portion of information 614a (e.g., wireless signals 624) indicative of the response of the second sensory prosthesis, and to generate a first comparison (e.g., first data 532a) of these first portions 514a, 614a. The second circuitry 530 can be further configured to, during a second time period, receive a second portion of information 514b (e.g., wireless signals 524) indicative of the response of the first sensory prosthesis, to receive a second portion of information 614b (e.g., wireless signals 624) indicative of the response of the second sensory prosthesis, and to generate a second comparison (e.g., second data 532b) of these second portions 514b, 614b.

The first data 532a generated by the second circuitry 530 can be indicative of a first frequency response difference between the at least one first transducer 510 and the at least one second transducer 610 and/or a first sensitivity difference between the at least one first transducer 510 and the at least one second transducer 610. Similarly, the second data 532b generated by the second circuitry 530 can be indicative of a second frequency response difference and/or a second sensitivity difference between the at least one first transducer 510 and the at least one second transducer 610.

For example, a frequency response difference at a time tₙ can be a difference between a frequency response R₁(tₙ) of the at least one first transducer 510 to the ambient sensory excitations 503 at time tₙ (e.g., distribution of acoustic frequencies detected by the at least one first microphone at time tₙ) and a frequency response R₂(tₙ) of the at least one second transducer 610 to the same ambient sensory excitations 503 at the time tₙ (e.g., distribution of acoustic frequencies detected by the at least one second microphone at time tₙ). For another example, the first sensitivity difference at a time tₙ can be a difference between a sensitivity S₁(tₙ) of the at least one first transducer 510 to the ambient sensory excitations 503 at time tₙ (e.g., amplitudes detected by the at least one first microphone at one or more predetermined acoustic frequencies to the ambient sound 103 at time tₙ) and a sensitivity S₂(tₙ) of the at least one second transducer 610 to the same ambient sensory excitations at the time tₙ (e.g., amplitudes detected by the at least one second microphone at the one or more predetermined acoustic frequencies to the same ambient sound 103 at time tₙ).

In certain implementations, the second circuitry 530 is configured to, prior to generating the first data 532a, evaluate whether the ambient sensory excitations 503 at the first time ty are sufficient for generation of the first data 532a. For example, the second circuitry 530 can evaluate whether the ambient sound 103 at the first time ty has sufficient amplitude and/or acoustic frequency range (e.g., sufficient amplitude at each frequency of a predetermined set of frequency ranges; sufficiently loud across a sufficiently wide frequency range) for generation of the first data 532a. Similarly, in certain implementations, the second circuitry 530 is configured to, prior to generating the second data 532b, evaluate whether the ambient sensory excitations 503 at the second time t₂ are sufficient for generation of the second data 532b. For example, the second circuitry 530 can evaluate whether the ambient sound 103 at the second time t₂ has sufficient amplitude and/or acoustic frequency range (e.g., sufficient amplitude at each frequency of a predetermined set of frequency ranges; sufficiently loud across a sufficiently wide frequency range) for generation of the second data 532b. If the amplitude and/or the acoustic frequency range at the first time t₁ are sufficient (e.g., greater than one or more predetermined threshold values), the second circuitry 530 can proceed to generating the first data 532a. If the amplitude and/or the acoustic frequency range at the first time t₁ are insufficient (e.g., less than the one or more predetermined threshold values), the second circuitry 530 halt the data generation process and can generate a warning signal (e.g., light; sound; text; image) to be received by the recipient and/or the practitioner. Similarly, if the amplitude and/or the acoustic frequency range at the second time t₂ are sufficient (e.g., greater than one or more predetermined threshold values), the second circuitry 530 can proceed to generating the second data 532b. If the amplitude and/or the acoustic frequency range at the second time t₂ are insufficient (e.g., less than the one or more predetermined threshold values), the second circuitry 530 halt the data generation process and can generate a warning signal (e.g., light; sound; text; image) to be received by the recipient and/or the practitioner.

In certain implementations, the second circuitry 530 is configured to generate the first data 532a upon receiving a first trigger signal. For example, the first trigger signal can comprise a first user input signal (e.g., via a button, touchscreen, or other user interface) indicative of a first command from the recipient and/or the practitioner to receive the first and second information 514a, 614a at the first time t₁ and to generate the first data 532a. The first trigger signal can be received by the second circuitry 530 during a fitting session in which the performance of the first and second sensory prostheses are being optimized by a practitioner for the recipient.

In response to the first trigger signal, the second circuitry 530 can sample the response of the at least one first transducer 510 to the ambient sensory excitations 503 at the first time t₁, sample the response of the at least one second transducer 610 to the same ambient sensory excitations 503 at the first time t₁, and calculate the first data 532a comprising a first frequency response difference at the first time t₁ (e.g., Δ_{R1}=|R₁(t₁)−R₂(t₁)|) and/or a first sensitivity difference at the first time t₁ (e.g., Δ_{S1}=|S₁(t₁)−S₂(t₁)|). The second circuitry 530 can be configured to, upon generating the first data 532a, store the first information 514a, the second information 614a, and/or the first data 532a for later retrieval for the performance evaluation.

In certain implementations, the second circuitry 530 is configured to generate the second data 532b upon receiving a second trigger signal. For example, the second trigger signal can comprise a second user input signal (e.g., via a button, touchscreen, or other user interface) indicative of a second command from the recipient and/or the practitioner to receive the first and second information 514b, 614b at the second time t₂ (e.g., subsequent to t₁) and to generate the second data 532b. The second trigger signal can be generated by the recipient and/or the practitioner during a subsequent fitting procedure in which the performance of the first and second sensory prostheses are being optimized by the practitioner for the recipient or at any other time at which the recipient and/or the practitioner thinks that a performance evaluation is warranted. For another example, the second trigger signal can comprise a clock signal indicative of passage of a scheduled time interval (e.g., one or more days; one or more weeks; one or more months) after generation of the first data 532a at the first time t₁. For another example, the second trigger signal can comprise a transducer signal (e.g., from the at least one first transducer 510 and/or the at least one second transducer 610) indicative of the ambient sensory excitations at the second time t₂ having predetermined attributes (e.g., aberrant amplitude and/or acoustic frequency range). The predetermined attributes can be determined by machine learning circuitry of the first sensory prosthesis, the second sensor prosthesis, and/or the device 650. For another example, the second trigger signal can comprise a signal generated by circuitry of the first and/or second sensory prosthesis (e.g., first circuitry 520; second stimulation circuitry 620), the signal indicative of differences between the responses of multiple transducers of the first and/or second sensory prosthesis (e.g., different microphones of the same acoustic prosthesis). For another example, the second trigger signal can comprise a sensor signal indicative of conditions that are potentially damaging to the at least one first transducer 510 and/or the at least one second transducer 610. The sensor signal can be provided by a sensor of the first and/or second sensory prosthesis (e.g., an accelerometer responsive to falls or impacts experienced by the recipient; a thermal sensor responsive to excessively hot and/or cold temperatures). For another example, the second trigger signal can result from a hardware reset of the circuitry of the first sensory prosthesis and/or the second sensory prosthesis.

In response to the second trigger signal, the second circuitry 530 can sample the responses of the at least one first transducer 510 to the ambient sensory excitations 503 at the second time t₂, sample the response of the at least one second transducer 610 to the same ambient sensory excitations 503 at the second time t₂, and calculate the second data 532b comprising a second frequency response difference at the second time t₂ (e.g., Δ_{R2}=|R₁(t₂)−R₂(t₂)|) and/or a second sensitivity difference at the second time t₂ (e.g., Δ_{S2}=|S₁(t₂)−S₂(t₂)|). The second circuitry 530 can be configured to, upon generating the second data 532b, store the first information 514b, the second information 614b, and/or the second data 532b for later retrieval and comparison to additional data.

In certain implementations, the performance evaluation 534 of the at least one aspect of the apparatus 500 (e.g., the at least one transducer 510, the first circuitry 520; the at least one second transducer 610, and/or the second stimulation circuitry 620) comprises comparing the first data 532a and the second data 532b. For example, the second circuitry 530 can retrieve the first data 532a from the storage circuitry and then compare the retrieved first data 532a to the second data 532b.

In certain implementations, the comparison of the first data 532a and the second data 532b is indicative of a difference between the first data 532a and the second data 532 b (e.g., Δ_{R}=|Δ_{R1}−Δ_{R2}; Δ_{S}=|Δ_{S1}−Δ_{S2}|) and the difference is compared to a predetermined threshold value. If the difference is less than the threshold value, then the second circuitry 530 of certain implementations provides a notification to the recipient and/or the practitioner (e.g., using light, sound, text, and/or image) that the performance evaluation did not detect an issue warranting further troubleshooting and/or investigation. Other example responses by the second circuitry 530 to the difference being less than the threshold include, but are not limited to, taking no action. If the difference is greater than the threshold value, then the second circuitry 530 of certain implementations provides a notification (e.g., using light, sound, text, and/or image) that an issue warranting further troubleshooting has been detected. Other example responses by the second circuitry 530 to the difference being greater than the threshold include, but are not limited to: sending the first data 532a and/or the second data 532b to the practitioner; scheduling a fitting session with the recipient and the practitioner; describing other actions to be taken (e.g., changing or cleaning the microphone covers).

In certain implementations, the predetermined threshold values comprise a static threshold value (e.g., in dB, for each frequency of a predetermined range of frequencies). The predetermined threshold values can be defined per unit time for specific combinations of sensory prostheses. For example, if a first sensory prosthesis reduces in sensitivity more slowly than a second sensory prosthesis, there would be a gradual increase in the difference of sensitivities over time, and the threshold values can be defined in dB/month or in dB/year. In certain implementations, the predetermined threshold values comprise a dynamic threshold value configured to be adjusted over time (e.g., as data is gathered from devices in the field). For example, a machine learning or statistical model can be used to take in the performance of many acceptable and unacceptable devices (e.g., including those returned for damage repair) and to output appropriate threshold values to be used.

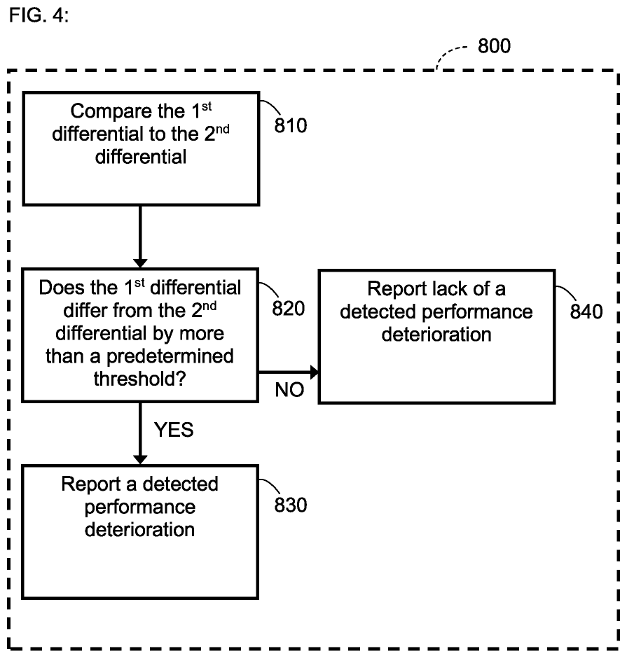

FIGS. 3A and 3B are flow diagrams of example methods 700a, b for performing an initial test at time tₙ (e.g., n=1) and a subsequent test at time tm (e.g., m>n) respectively, in a performance evaluation of a sensory prosthesis system (e.g., bilateral acoustic prosthesis system) in accordance with certain implementations described herein. For example, the initial test at time tₙ can be the first test ever performed for the sensory prosthesis system (e.g., as part of a fitting or programming procedure for the sensor prosthesis system at the practitioner's facility under controlled conditions) and the subsequent test at time tm can be an “in-the-field” test performed during the normal operation of the sensory prosthesis system (e.g., the test performed under non-controlled conditions). For another example, the initial test at time tₙ can be performed during the normal operation of the sensory prosthesis system (e.g., the test performed under non-controlled conditions) and the subsequent test at time tm can be performed later than the initial test and during the normal operation of the sensory prosthesis system (e.g., performed under non-controlled conditions). FIG. 4 is a flow diagram of an example method 800 for detecting a performance degradation of one of the first and second sensory prostheses of a sensory prosthesis system (e.g., bilateral acoustic prosthesis system) in accordance with certain implementations described herein.

While the example methods 700a,b, 800 are described herein by referring to the example apparatus 500 of FIGS. 2A-2B, other apparatuses are also compatible with the example methods 700a,b, 800 in accordance with certain implementations described herein. For example, the methods 700a,b, 800 described herein can be applied to any of a variety of sensory prosthesis systems having multiple transducers.

In an operational block 710a, the method 700a comprises initiating the initial test. For example, the initial test can be initialized in response to a first trigger signal from a user (e.g., the recipient and/or the practitioner) or in response to a first automated trigger signal (e.g., generated by the second circuitry 530 as part of a fitting or programming procedure for the sensory prosthesis system). In certain implementations in which the initial test is performed as part of a fitting or programming procedure for the sensory prosthesis system (e.g., at a practitioner's facilities), initiating the initial test can further comprise emitting a predetermined sensory excitation into the ambient environment (e.g., sound from a speaker) to be detected by the at least one first transducer 510 and the at least one second transducer 620. To increase effectiveness, the predetermined sound can be delivered in a prescribed matter. For example, the predetermined sound can be delivered from a speaker that is equally distant from both the at least one first transducer 510 and the at least one second transducer 610 so that variations in the measurements (e.g., in the operational block 730a) are due to differences in the at least one first and second transducers 510, 610 and/or the sensory prostheses, rather than differences in the distance between the speaker and the at least one first and second transducers 510, 610.

In an operational block 720a, the method 700a further comprises assessing the ambient sensory stimulation (e.g., ambient sound) against predetermined suitability criteria for suitability for use in the initial test. Examples of predetermined suitability criteria include, but are not limited to: broadband power level (e.g., not too low or too high); shape of the frequency spectrum; associated metadata to understand the suitability of the ambient sound environment (e.g., scene classifier state; directionality of signals); data from additional sensors (e.g., accelerometer data to determine if the recipient's head is moving). For example, ambient sound can be assessed by comparing the ambient sound as detected by the at least one first transducer 510 (e.g., microphone) and/or the at least one second transducer 610 (e.g., microphone) against predetermined thresholds of sufficient amplitude and/or acoustic frequency range. If the ambient sound is assessed to be suitable (e.g., the amplitude and/or the acoustic frequency range at the time tₙ being greater than corresponding predetermined threshold values), the initial test can proceed. If the ambient sound is assessed to be unsuitable (e.g., the amplitude and/or the acoustic frequency range at the time tₙ being less than corresponding predetermined threshold values), the initial test can be paused and the ambient sound can continue to be monitored and assessed until the ambient sound satisfies the suitability criteria, and the initial test can then proceed.

In an operational block 730a, the method 700a further comprises, at time tₙ, measuring the ambient sensory stimulation using the at least one first transducer 510 and using the at least one second transducer 620. The measurements at time tₙ are indicative of the responses of the transducers 510, 610 to the same ambient sensory excitations being received by the transducers 510, 610 at time tₙ. In an operational block 740a, the method 700a further comprises analyzing the measurements at time tₙ to determine characteristics (e.g., frequency response; sensitivity) of the responses of the transducers 510, 610. In certain implementations, each measurement is analyzed by the sensory prostheses that performed the measurement, while in certain other implementations, both measurements are analyzed by the same sensory prosthesis and/or by a separate device 650. In an operational block 750a, the method 700a further comprises measuring and storing a comparison of the transducer responses at time tₙ (e.g., a first differential between the characteristics of the responses at time tₙ of the transducers 510, 610).

For performing the subsequent test at time tₘ (e.g., m>n), in an operational block 710b, the method 700b comprises initiating the subsequent test. For example, the subsequent test can be initialized in response to a second trigger signal from a user (e.g., the recipient and/or the practitioner) or in response to a second automated trigger signal. The second automated trigger signal can be automatically generated by the second circuitry 530 in response to at least one of: a predetermined amount of time elapsed since the time tₙ; a detected potentially damaging event (e.g., a device impact detected by an accelerometer; a temperature extreme detected by a thermal sensor); a detected change in comparison data between multiple transducers within a single sensory prosthesis. Unlike the initial test, the subsequent test of certain implementations does not utilize any predetermined sensory excitations.

In an operational block 720b, the method 700b further comprises assessing the ambient sensory stimulation against the predetermined suitability criteria for suitability for use in the subsequent test. For example, similar to the assessing in the operational block 720a, ambient sound can be assessed by comparing the ambient sound as detected by the at least one first transducer 510 (e.g., microphone) and/or the at least one second transducer 610 (e.g., microphone) against predetermined thresholds of sufficient amplitude and/or acoustic frequency range. If the ambient sound is assessed to be suitable (e.g., the amplitude and/or the acoustic frequency range at the time tm being greater than corresponding predetermined threshold values), the subsequent test can proceed. If the ambient sound is assessed to be unsuitable (e.g., the amplitude and/or the acoustic frequency range at the time tₘ being less than corresponding predetermined threshold values), the subsequent test can be paused and the ambient sound can continue to be monitored and assessed until the ambient sound satisfies the suitability criteria, and the subsequent test can then proceed.

In an operational block 730b, the method 700b further comprises, at time tₘ, measuring the ambient sensory stimulation using the at least one first transducer 510 and using the at least one second transducer 620. The measurements at time tm are indicative of the responses of the transducers 510, 610 to the same ambient sensory excitations being received by the transducers 510, 610 at time tₘ. In an operational block 740b, the method 700b further comprises analyzing the measurements at time tₘ to determine characteristics (e.g., frequency response; sensitivity) of the responses of the transducers 510, 610. In certain implementations, each measurement is analyzed by the sensory prostheses that performed the measurement, while in certain other implementations, both measurements are analyzed by the same sensory prosthesis and/or by a separate device 650. In an operational block 750b, the method 700b further comprises measuring and storing a comparison of the transducer responses at time tm (e.g., a second differential between the characteristics of the responses at time tm of the transducers 510, 610).

In an operational block 810, the method 800 comprises comparing the first differential (e.g., previously measured and stored during the operational block 750a) to the second differential (e.g., previously measured and stored during the operational block 750b). For example, a difference (e.g., an absolute value of the difference) between the first differential and the second differential can be calculated. In certain implementations in which the short-term variation in the ambient sound environment (e.g., sound coming from one side of the recipient) can interfere with this comparison, to compensate, each measurement can be defined as an average of the data collected by the transducer over a period of time (e.g., multiple hours).

In the operational block 820, the method 800 further comprises determining whether the calculated difference between the first and second differentials is greater than a predetermined threshold value. Depending on how the threshold value is defined, the calculated differential can be compared in various ways. For example, the latest measurement Cn can be compared to the initial measurement Co, which can be useful to identify gradual drifts over time. For another example, the latest measurement Cn can be compared to the previous measurement Cn-1, which can be useful to identify relatively sudden changes in performance. For another example, the latest measurement Cn can be compared to the trendline established by all or several of the measurements Co to Cn−1, which can be useful to identify a trend (e.g., using the gradient of a curve) even if the performance has not yet deteriorated enough to reach a single defined threshold.

The predetermined threshold value can be determined in various ways in accordance with certain implementations described herein. For example, the predetermined threshold value can be a static defined threshold (e.g., in dB, for each frequency of a range of frequencies). For another example, the predetermined threshold value can be a static defined threshold defined per unit time (e.g., in dB/month or dB/year). Such a threshold value can be useful for specific combinations of sensory prostheses that have sensitivities with different speeds of reduction, which could produce a gradual difference in sensitivity over time. For another example, the predetermined threshold value can be a dynamically defined threshold (e.g., adjusted over time, as the manufacturer gathers data from devices in the field). Such dynamically defined thresholds can be the output of machine learning or statistical modeling that takes as input the performance of many acceptable and unacceptable devices (e.g., including those returned for repair as damaged). For another example, the predetermined threshold value can be a dynamically defined threshold that is dependent on the combination of devices used in the comparison (e.g., whether the devices are on the same side of the head). For another example, the predetermined threshold value can be a dynamically defined threshold that is dependent on previous clinical testing (e.g., if the algorithm for a particular recipient indicates many false alarms when there is no actual hardware issue, the threshold can be increased).

If the calculated difference is greater than the predetermined threshold value, the method 800 further comprises, in an operational block 830, generating a report signal indicative of a detected performance deterioration. If the calculated difference is not greater than the predetermined threshold value, the method 800 further comprises, in an operational block 840, generating a report signal indicative of a lack of a detected performance deterioration.

In certain implementations, multiple comparisons can be performed during each performance evaluation. A performance deterioration detected by any of the performance evaluations can indicate an issue with at least one of the transducers 510, 610 and/or the first and second sensory prostheses and could be used to initiate further testing and/or investigation. If at least one prosthesis comprises more than one transducer (e.g., microphone), the multiple comparisons can include comparisons of different combinations of the transducers (e.g., compare a first microphone of the first prosthesis to a first microphone of the second prosthesis; compare a second microphone of the first prosthesis to the first microphone of the second prosthesis; etc.). If there are two or more prostheses in the system, at least one of which has multiple transducers (e.g., a recipient with a left-side cochlear implant with an implanted microphone and a left-side sound processor with external microphones and a right-side hearing aid) and a non-prosthesis device (e.g., smartphone) with a transducer (e.g., microphone), the multiple comparisons can include comparison of a first microphone of the first prosthesis to a first microphone of the second prosthesis; compare the first microphone of the first prosthesis to a first microphone of the non-prosthesis device; etc. In certain implementations in which not all comparisons are equivalent (e.g., a comparison between an implanted microphone and a non-implanted microphone on the same side of the recipient's head that are not affected by the direction from which an ambient sound is received), weighting can be applied so that certain combinations of devices have a larger impact on the overall evaluation.

In certain implementations, a transform function can be applied to the measurements from one of the devices, the transform function depending on the types of devices to be compared. For example, an implanted microphone can experience a different sound environment due to the overlying tissue layers than a non-implanted microphone. A transform function can be applied to the measurements of one of the devices (e.g., the implanted microphone) before performing the comparison in order to better match the data output by the other device.

Although commonly used terms are used to describe the systems and methods of certain implementations for ease of understanding, these terms are used herein to have their broadest reasonable interpretations. Although various aspects of the disclosure are described with regard to illustrative examples and implementations, the disclosed examples and implementations should not be construed as limiting. Conditional language, such as, among others, “can,” “could,” “might,” or “may,” unless specifically stated otherwise, or otherwise understood within the context as used, is generally intended to convey that certain implementations include, while other implementations do not include, certain features, elements and/or steps. Thus, such conditional language is not generally intended to imply that features, elements and/or steps are in any way required for one or more implementations or that one or more implementations necessarily include logic for deciding, with or without user input or prompting, whether these features, elements and/or steps are included or are to be performed in any particular implementation. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

It is to be appreciated that the implementations disclosed herein are not mutually exclusive and may be combined with one another in various arrangements. In addition, although the disclosed methods and apparatuses have largely been described in the context of auditory prostheses, various implementations described herein can be incorporated in a variety of other suitable devices, methods, and contexts. More generally, as can be appreciated, certain implementations described herein can be used in a variety of implantable medical device contexts that can benefit from having two devices in or on the recipient's body, each with one or more transducers making measurements of the same aspect of the ambient environment or of the recipient's body.

Language of degree, as used herein, such as the terms “approximately,” “about,” “generally,” and “substantially,” represent a value, amount, or characteristic close to the stated value, amount, or characteristic that still performs a desired function or achieves a desired result. For example, the terms “approximately,” “about,” “generally,” and “substantially” may refer to an amount that is within ±10% of, within ±5% of, within ±2% of, within ±1% of, or within ±0.1% of the stated amount. As another example, the terms “generally parallel” and “substantially parallel” refer to a value, amount, or characteristic that departs from exactly parallel by ±10 degrees, by ±5 degrees, by ±2 degrees, by ±1 degree, or by ±0.1 degree, and the terms “generally perpendicular” and “substantially perpendicular” refer to a value, amount, or characteristic that departs from exactly perpendicular by ±10 degrees, by ±5 degrees, by ±2 degrees, by ±1 degree, or by ±0.1 degree. The ranges disclosed herein also encompass any and all overlap, sub-ranges, and combinations thereof. Language such as “up to,” “at least,” “greater than,” less than,” “between,” and the like includes the number recited. As used herein, the meaning of “a,” “an,” and “said” includes plural reference unless the context clearly dictates otherwise. Also, as used in the description herein, the meaning of “in” includes “into” and “on,” unless the context clearly dictates otherwise.

While the methods and systems are discussed herein in terms of elements labeled by ordinal adjectives (e.g., first, second, etc.), the ordinal adjective are used merely as labels to distinguish one element from another (e.g., one signal from another or one circuit from one another), and the ordinal adjective is not used to denote an order of these elements or of their use.

The invention described and claimed herein is not to be limited in scope by the specific example implementations herein disclosed, since these implementations are intended as illustrations, and not limitations, of several aspects of the invention. Any equivalent implementations are intended to be within the scope of this invention. Indeed, various modifications of the invention in form and detail, in addition to those shown and described herein, will become apparent to those skilled in the art from the foregoing description. Such modifications are also intended to fall within the scope of the claims. The breadth and scope of the invention should not be limited by any of the example implementations disclosed herein, but should be defined only in accordance with the claims and their equivalents.

1. An apparatus comprising:
at least one first transducer configured to be worn on or within a recipient's body, the at least one first transducer configured to generate transducer signals indicative of ambient sound from an environment of the recipient;
first circuitry configured to receive the transducer signals and, in response to the transducer signals, to generate stimulation signals configured to be received by the recipient's body to evoke a hearing percept by the recipient, the stimulation signals indicative of the ambient sound; and
second circuitry configured to:
generate first data indicative of a first comparison of a response of the at least one first transducer to the ambient sound at a first time to a response of at least one second transducer to the ambient sound at the first time;
generate second data indicative of a second comparison of a response of the at least one first transducer to the ambient sound at a second time to a response of the at least one second transducer of the device to the ambient sound at the second time, the second time subsequent to the first time; and
generate a performance evaluation of at least one aspect of the apparatus in response to a third comparison of the first data and the second data.

2. The apparatus of claim 1, wherein the at least one first transducer and the first circuitry are components of a first auditory prosthesis in operable communication with a first auditory subsystem of the recipient and the at least one second transducer is a component of a second auditory prosthesis separate from the first auditory prosthesis and in operable communication with a second auditory subsystem of the recipient.

3. (canceled)

4. (canceled)

5. (canceled)

6. (canceled)

7. The apparatus of claim 1, wherein the first data is indicative of a first frequency response difference between the at least one first transducer and the at least one second transducer and/or a first sensitivity difference between the at least one first transducer and the at least one second transducer and the second data is indicative of a second frequency response difference between the at least one first transducer and the at least one second transducer and/or a second sensitivity difference between the at least one first transducer and the at least one second transducer.

8. The apparatus of claim 1, wherein the third comparison comprises a comparison of a difference between the first data and the second data to a predetermined threshold.

9. The apparatus of claim 1, wherein the second circuitry is configured to evaluate whether the ambient sound at the second time is suitable for generation of the second data.

10. The apparatus of claim 1, wherein the second circuitry is configured to generate the second data in response to: passage of a predetermined time interval after generation of the first data, the ambient sound at the second time having predetermined attributes, and/or the second circuitry receiving sensor signals indicative of conditions that are potentially damaging to the at least one first transducer.

11. The apparatus of claim 1, wherein the second circuitry is configured to generate the first data by receiving a first portion of the transducer signals at the first time and receiving information indicative of a first response of the at least one second transducer at the first time, the first comparison comprising a comparison of the first portion of the transducer signals at the first time and the information indicative of the first response of the at least one second transducer at the first time.

12. The apparatus of claim 11, wherein the second circuitry is configured to generate the second data by receiving a second portion of the transducer signals at the second time and receiving information indicative of a second response of the at least one second transducer at the second time, the second comparison comprising a comparison of the second portion of the transducer signals at the second time and the information indicative of the second response of the at least one second transducer at the second time.

13. The apparatus of claim 1, wherein the performance evaluation comprises a performance evaluation of the at least one first transducer.

14. An apparatus comprising:
communication circuitry configured to receive first information from at least one first transducer and second information from at least one second transducer, the first information and the second information indicative of signals from an environment of a recipient or from the recipient's body; and
evaluation circuitry configured to, during a first time period:
receive a first portion of the first information indicative of a response of the at least one first transducer to the signals during the first time period;
receive a first portion of the second information indicative of a response of the at least one second transducer to the signals during the first time period; and
generate a first comparison of the first portion of the first information and the first portion of the second information;
the evaluation circuitry further configured to, during a second time period subsequent to the first time period:
receive a second portion of the first information indicative of a response of the at least one first transducer to the signals during the second time period;
receive a second portion of the second information indicative of a response of the at least one second transducer to the signals during the second time period;
generate a second comparison of the second portion of the first information and the second portion of the second information; and
the evaluation circuitry further configured to generate a third comparison of the first comparison and the second comparison.

15. The apparatus of claim 14, wherein the evaluation circuitry is further configured to evaluate, in response to the third comparison, performance of at least one of the at least one first transducer and the at least one second transducer.

16. The apparatus of claim 14, further comprising a smartphone, tablet, or computer comprising the communication circuitry and the evaluation circuitry.

17. The apparatus of claim 14, wherein the at least one first transducer and the at least one second transducer are both in operative communication with the same sensory prosthesis.

18. The apparatus of claim 14, wherein the at least one first transducer is a component of a first sensory prosthesis and the at least one second transducer is a component of a second sensory prosthesis different from the first sensory prosthesis.

19. The apparatus of claim 14, further comprising stimulation circuitry configured to generate and provide stimulation signals to the recipient's body in response to the first information and the second information, the signals comprising ambient sensory excitations from an environment of the recipient.

20. The apparatus of claim 14, wherein the signals are generated by or are indicative of properties of the recipient's body.

21. A method comprising:
measuring and storing a first differential between responses of a first prosthesis and a second prosthesis to ambient excitations at a first time;
measuring and storing at least one second differential between responses of the first prosthesis and the second prosthesis to ambient excitations at a second time subsequent to the first time; and
detecting a performance degradation of one of the first and second prostheses, said detecting comprising comparing the at least one first differential to the second differential.

22. (canceled)

23. (canceled)

24. The method of claim 21, wherein said detecting the performance degradation further comprises calculating a difference between the first differential and the second differential and determining whether the calculated difference is greater than a predetermined threshold value.

25. The method of claim 24, further comprising generating a signal indicative of a detected performance deterioration if the calculated difference is greater than the predetermined threshold value.

26. The method of claim 24, further comprising generating a signal indicative of a lack of a detected performance deterioration if the calculated difference is not greater than the predetermined threshold value.

27. (canceled)

28. (canceled)

29. (canceled)

30. (canceled)
